(19) United States
(12) Patent Application Publication
(10) Pub. No.: US 2010/0114264 A1
(43) Pub. Date: May 6, 2010

(54) DEVICE FOR IRRADIATING AN OBJECT, IN PARTICULAR HUMAN SKIN, WITH UV LIGHT

(76) Inventor: Andreas LECHTHALER, Nenzing (AT)

Correspondence Address: WENDEROTH, LIND & PONACK, L.L.P., 1030 15th Street, N.W.,, Suite 400 East, Washington, DC, 20005-1503, US

(21) Appl. No.: 12/636,061

(22) Filed: Dec. 11, 2009

Related U.S. Application Data
(63) Continuation of application No. PCT/AT2008/000205, filed on Jun. 12, 2008.

(30) Foreign Application Priority Data
Jun. 13, 2007 (AT) A 917/2007

(51) Int. Cl. A61N 5/06

(52) U.S. Cl. 607/88

(57) ABSTRACT

The invention relates to a device for irradiating an object, in particular human skin, with UV light. Said device comprises a UV light source and an irradiation head containing imaging optics, UV light being projected from the irradiation head onto the object. A light source that emits visible light is provided in addition to the UV light source, the light from said additional source being projected onto the object by means of the imaging optics of the irradiation head. According to the invention, a preferably electronically controlled unit for the variable adjustment of the light distribution on the object is located in the irradiation head and UV light from the UV light source and/or visible light from the light source emitting visible light can be selectively or simultaneously supplied to said unit.

The invention relates to a device for irradiating an object, in particular human skin, with UV light, having a UV light source and an irradiation head which contains imaging optics from which UV light is projected onto the object. In order to facilitate aligning the irradiation head relative to the irradiated object, and, possibly, to be able to more easily establish the definition of partial regions to be irradiated, the invention provides that in addition to the UV light source a light source which emits visible light is provided, the light of which can be projected onto the object via the imaging optics of the irradiation head, wherein arranged in the irradiation head is a means which is preferably electronically controlled for adjustably setting the light distribution on the object, and that means can optionally or simultaneously be subject to UV light from the UV light source and/or to visible light from the light source which emits visible light.

If, for example, a grid is projected onto the object, which is generally curved, by minimizing the distortion whilst the grid is being viewed it is easy and simple to align the irradiation head properly relative to the object.

It is also possible to define (e.g. by penning) on a screen, for example, partial regions of the surface which is to be treated, and to project that image on the screen, or a correlated image, directly onto the object, or human skin. By comparing the screen and object, constant monitoring is possible.

Further advantages and characteristics of the invention will be described with the aid of the following drawings.

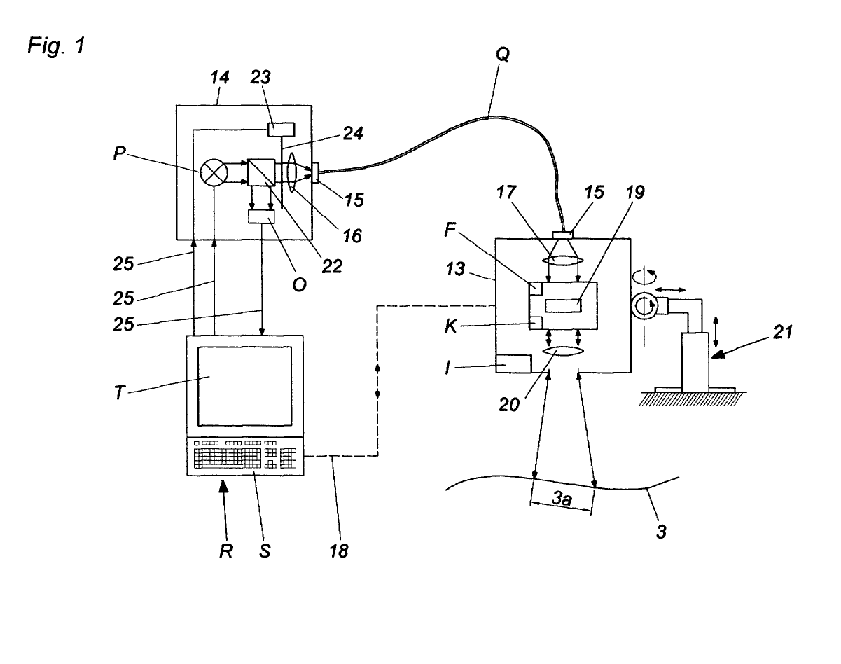

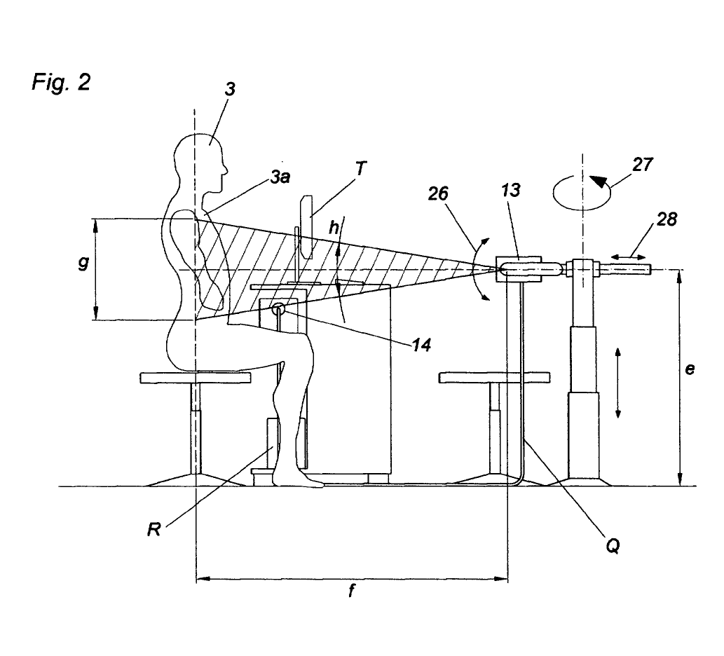

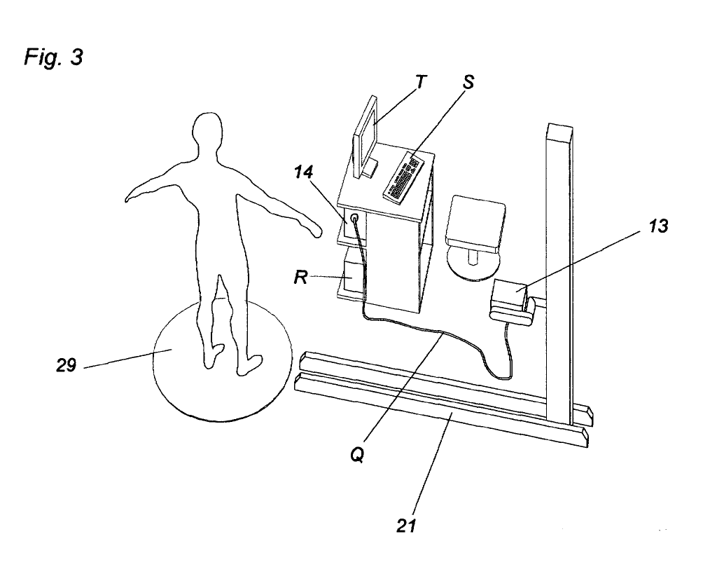

FIG. 1 is a diagrammatic representation of an embodiment of a device according to the invention. FIG. 2 shows another embodiment which is suitable for ambulant use. FIG. 3 shows an embodiment which is particularly suitable for stationary use, e.g. in a clinic.

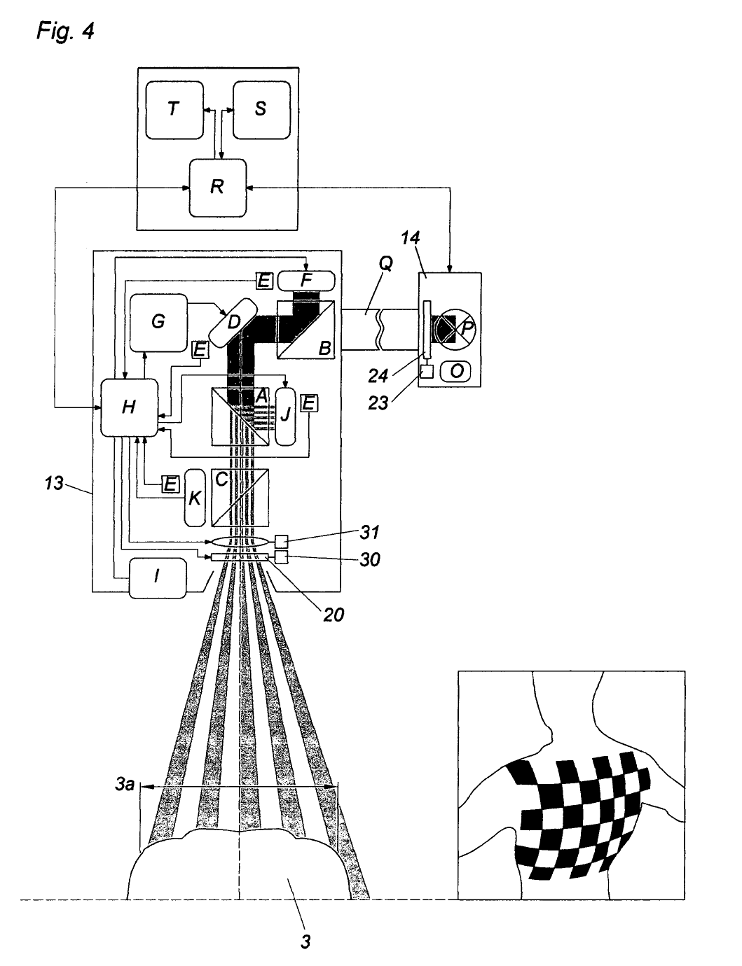

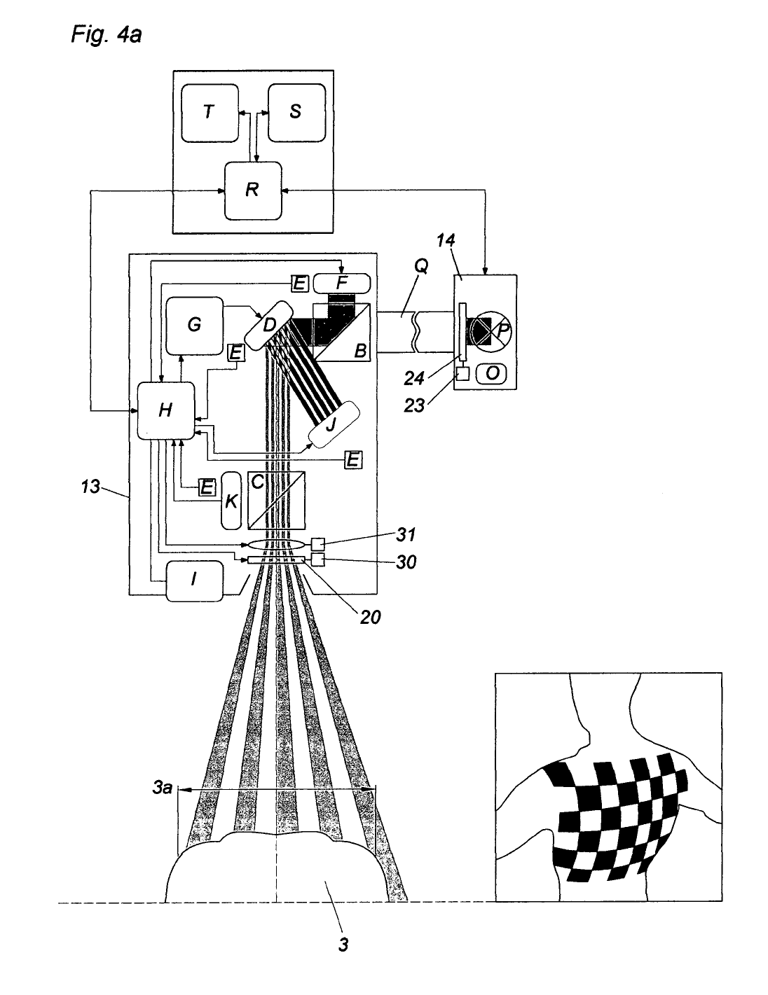

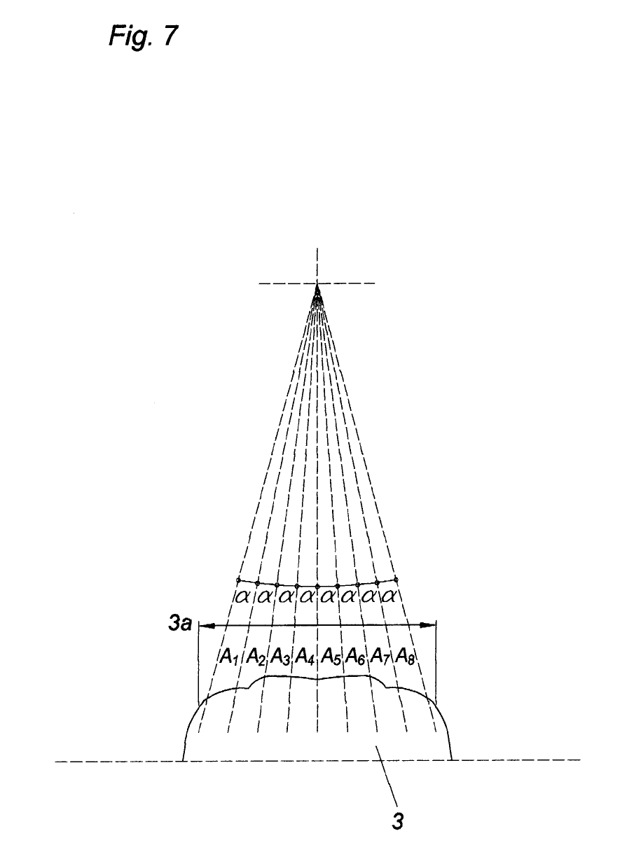

FIGS. 4, 5, 6, 8, 9, 10 and 11 are schematic representations of various operating states of another embodiment of the invention (with LCOS modulator). FIG. 7 is a view describing the spatial detection of the course of the surface of the object, in particular human skin. FIGS. 4a, 5a, 6a, 8a, 9a, 10a and 11a are schematic representations of various operating states of another embodiment of the invention corresponding to FIGS. 4, 5, 6, 8, 9, 10 and 11, but with a DLP or DMD modulator.

In the embodiment shown in FIG. 1, a UV light source P is provided which is housed, according to the invention, outside the irradiation head 13 in a separate light source housing 14. Arranged between the light source housing 14 and the irradiation head 13 there is at least one flexible fiber optic cable Q via which UV light can be fed from the UV light source P to the irradiation head 13.

The flexible fiber optic cable can contain at least one quartz glass fiber for low-loss UV light conduction. In order to protect the flexible optical fiber, it can be enclosed in a light-proof manner.

In order to be able to exchange individual components with greater ease, according to one preferred embodiment it can be provided that the flexible fiber optic cable is connected by means of a releasable connection 15 to the UV light source housing 14 or the irradiation head 13.

The UV light is input into the optical fiber via an input collimating lens 16, and is output from the irradiating head 13 via an output collimating lens 17.

To control the individual components a control computer R is provided which has a keyboard S or other input device, in particular a computer mouse and/or a lightpen/graphics tablet, etc. The control computer R has a screen (DFD, plasma, CRD) or a holographic projector as display device. In the present example, shown in FIG. 1, the control computer is a laptop or a notebook.

A means which is preferably electronically controllable via connections 18 for adjustably setting the light distribution on the object, or, to be more exact, on the surface 3a of the object to be irradiated, can be arranged in the irradiation head 13. This means is only shown diagrammatically in FIG. 1 and is denoted by the reference numeral 19. A means such as this, which will be described again in greater detail with the aid of the following embodiments, permits the irradiation at varying intensities selectively of partial regions of the region 3a of the object 3 which is to be irradiated, which is a great advantage when treating a variety of skin disorders, since in this way it is possible to adapt the intensity of the radiation properly to the local affected region. In so doing, the light passes out of the irradiation head 13 via the imaging optics 20, shown only diagrammatically as one lens, but which in practice can also comprise a plurality of lenses.

The irradiation head can also have a light source F which emits visible light, shown only diagrammatically in FIG. 1. By means of this light source it is possible to project a visible image onto the skin. A camera, preferably a CCD-camera K which delivers electrical image signals, can also be disposed in the irradiation head 13. This camera can firstly—as will be explained in greater detail hereinafter—receive light from the UV light source P or from the colored light source F, which is mainly of importance for calibration purposes. During its operation, the CCD-camera K can, however, also take images of the portion 3a which is to be irradiated, and detect the UV light which is reflected by the surface 3a during irradiation. This will be explained in greater detail hereinafter.

A means I for detecting the distance away and/or spatial course of the surface 3a of the object can also be disposed on the irradiation head 13. This means makes it possible to accurately set the intensities which actually reach the partial regions of the surface 3a. The intensity is actually dependent not only upon the energy which is radiated within the region of a specific solid angle, but also upon the surface of the partial region which is being irradiated. That surface is dependent, in turn, upon the distance away and spatial course of the surface of the object. If the geometric course is known, then—as will be explained in greater detail hereinafter—it is possible to correct the amount of energy in the individual solid angle regions, so that the desired intensity is, in fact, produced on the surface to be irradiated. This may even occur in a dynamic way, e.g. when the patient breathes and the surface 3a thus moves.

In FIG. 1 there is also a carrier device, denoted generally by the reference numeral 21, for the irradiation head 13. The irradiation head 13 can be mounted displaceably and/or rotatably on the carrier device, in order to achieve optimum alignment relative to the surface to be irradiated. It is also possible for the irradiation head to be adjusted by means of a motor.

A photospectrometer O supplied with light from the UV light source P via a beam splitter 22 can be provided in the light source housing 14 in order to be able to detect the spectral light distribution of the UV light of the UV source P.

Finally, an exit shutter 24, which is preferably movable by means of a motor 23, can be provided in the light source housing. By virtue of this exit shutter 24, the emergence of light into the light conductor, and thus the irradiation head, can be prevented if no UV light is required there, even when the UV light source P is switched on.

The UV light source housing 14 communicates overall with the control computer R by means of connections 25, which can also be grouped together to form a bus system.

FIG. 2 shows an embodiment of a device according to the invention which is suitable for ambulant use. Parts which are the same as in FIG. 1 are denoted by the same reference numerals. A region 3a can be identified above the irradiation head. The angles of opening h produce the size of the irradiation window g. The distance is denoted by the reference f.

The irradiation head 13 can be moved linearly in telescopic manner through the height e. The irradiation head 13 can also be adjusted in respect of its elevation angle (arrow 26) and in respect of its azimuth angle (arrow 27). Linear displacement horizontally (arrow 28) is also possible. Finally, the irradiation head 13 can also be rotated, preferably through 90°, about the optical axis, indicated by a broken line, which leads to the patient. Therefore, a rectangular irradiation surface can be transformed from portrait format to landscape format (and vice versa). Therefore, the irradiation head 13 can be aligned in the optimum position relative to the object (patient 3) who is sitting on a chair in the present example.

In the embodiment shown in FIG. 3, the irradiation head 13 is likewise mounted adjustably on a carrier device 21. This latter has two linear axes which are adjustable by means of motors in the vertical and horizontal directions. The rotational mounting of the irradiation head 13 can also be adjusted by means of a motor. That adjustment takes place by means of the control computer R which communicates with the servo motors in a way which is not shown.

Contrary to the embodiment of FIG. 2, the object, or the patient itself, is able to move in the embodiment of FIG. 3, since he stands on a rotating platform 29 which is controlled by the control computer R. Therefore, to align the irradiation head 13 and the patient 3 relative to each other, not only does the irradiation head move, but so does the patient as well.

In the following drawings, parts which are the same or equivalent to those in the earlier drawings are denoted by the same reference numerals.

The irradiation head 13 is shown in greater detail in the embodiment of FIG. 4. However, optical details, such as the collimating lens, which are not needed for the purpose of aiding comprehension, are omitted for the sake of simplicity. Structurally, the construction of the overall installation is similar to that of FIG. 1. There is a housing 14 for the UV light source P which communicates with the actual irradiation head via a flexible light cable Q. The electronic components of the control computer R, together with the keyboard S and screen T, are likewise arranged separately, and communicate via connections, or a bus system, firstly with the irradiation head 13, and secondly with the UV light source housing 14.

By virtue of the beam splitter B (preferably a dichroitic prism), on the one hand light from the UV light source P via the fiber optic cable Q and, on the other hand, light from a colored light source F can reach the other components of the irradiation head, or the object 3a.

In the embodiment shown in FIG. 4, the apparatus is in positioning-mode or Teach-In-Mode. Therein, the shutter 24 of the UV light source P is closed, or the UV light source is switched off. On the other hand, the light source F which emits visible light is switched on. This light source F can preferably be an RGB unit which contains light diodes and which can emit both colored and white light. For the present adjustment operation, colored light, e.g. red light, is emitted. The light source F is controlled by the electronic control unit (control computer R) by way of a (sub-) control unit, e.g. FGPA or DSP, arranged in the irradiation head 13. A temperature monitoring sensor E monitors the temperature of the visible light-emitting RGB light source F. Light from the light source F reaches the electronically controllable modulator for spatial light D (EASLM) via the beam splitter B. The modulator can be a Liquid Crystal on Silicon-Unit (LCOS), for example. The modulator D is controlled by the control unit H by way of an image data preparation unit G. Subject to control by the modulator D, light reflected by this latter then reaches, depending upon the polarization, either the dichroitic prism C via splitter prism A with polarizing filter, or it reaches a cooling member J which receives the light which is not intended for the prism C but which is intended to reach the object to be treated.

By way of the light modulator D, which, like other components, can be monitored by temperature sensors E, it is possible, e.g. in an imaginary pixel grid, to illuminate certain zones on the object, namely with variable brightness or intensity, and others not. Lastly, the modulator D forms the core piece for the selective radiation of partial regions on the object to be irradiated.

In accordance with the mode of operation shown in FIG. 4 for the handling setup, the modulator D is controlled in such a way that a relatively large chequered pattern results on the object (see FIG. 4, bottom right). In that operating state, the exit shutter 30 is opened by means of the motor 31. The imaging optics 20 is preferably continuously adjustable by means of motors (m) by the control unit H so as to perform a zooming and focusing function. After the zoom and focus have been set (which is evident by a sharp image of the chequered pattern on the object) the irradiation head can be aligned relative to the patient, or object, so that in the active irradiation window the trapezoidal and pincushion distortion caused by the generally curved course of the object is minimal. The camera K and 3-D scanner I, yet to be described hereinafter, are not active. The irradiation head simply undergoes an initial adjustment relative to the patient here.

After that initial adjustment is complete, all the relevant adjustment parameters can then be stored, e.g. in files in the control computer R relating to the patient or treatment. During a further session, those files can be called up again, thus permitting rapid initial adjustment.

FIG. 4a shows another embodiment in the same operating mode as in FIG. 4. Contrary to the LCOS unit D whose operation is polarization-based, in the embodiment of FIG. 4a a DLP (Digital Light Processing) unit is provided. This can, for example, be a Digital Micromirror Device (DMD) which is accommodated on a chip. A DMD-chip of this kind has, distributed over the surface thereof, microscopically small mirrors, the edge length of which can be within the range of 10 μm. These mirrors can be controlled electronically, e.g. by electrostatic fields, in respect of their alignment. By inclining the individual micromirrors on the DMD chip D the light is reflected either directly to the beam splitter C and then on to the patient, or to the absorber J. By pulse-width modulated control of the mirrors it is possible to produce varying degrees of brightness of the individual image points. In other respects, the construction is the same as the LCOS-variant of FIG. 4.

As shown in FIGS. 2 and 3, the screen D is, in practice, arranged so that it can be viewed by the person looking at it, e.g. the doctor, as well as being within the view of the irradiated region of the object 3. It is therefore possible to view simultaneously the image displayed on the screen and a correlated image on the object which has been generated by the modulator from the colored light source F. This is very advantageous for checking purposes.

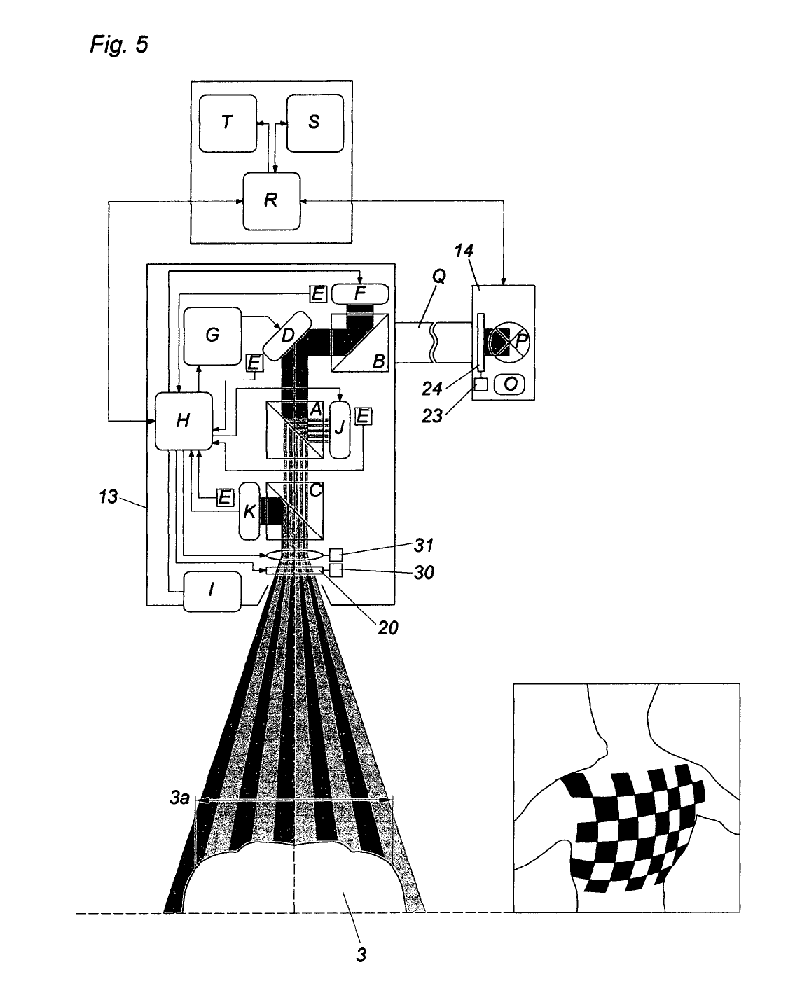

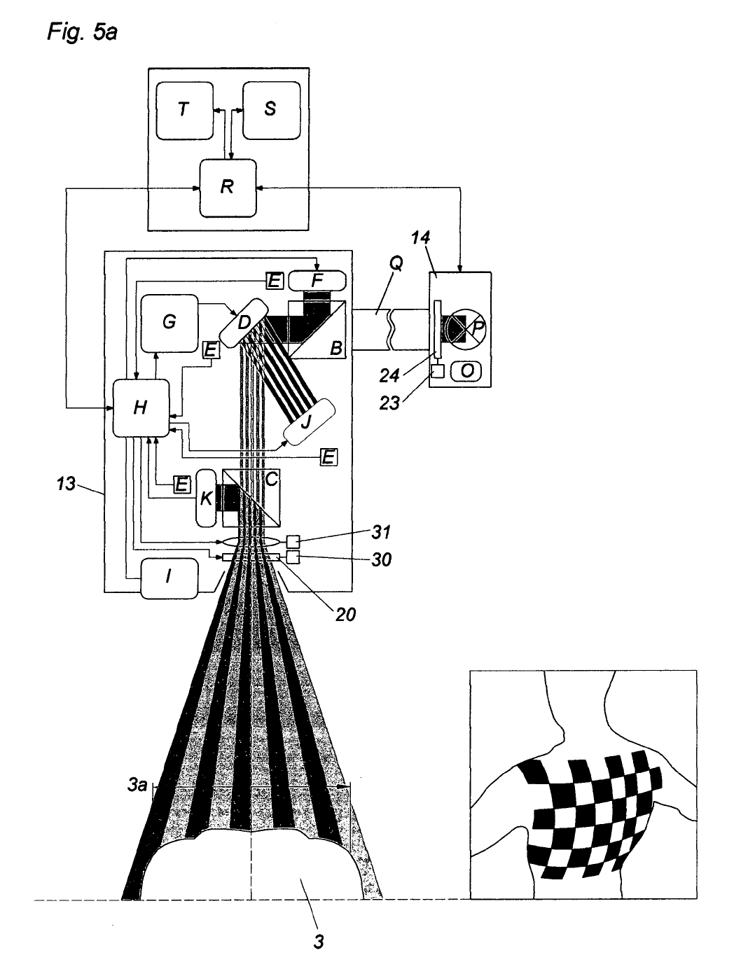

FIG. 5 shows the same means as FIG. 4, but in a different operating mode—namely the detection of an image of the region (3a) of the object which is to be treated during the next set up step in the process. For this purpose, in the irradiation head 13 there is a camera K, preferably a CCD camera. This emits electrical image signals to the control unit H and then on to the control computer R.

After correct positioning has been completed, FIG. 4, completion is confirmed in the control computer R by means of a control element or input element S. The modulator D is then activated automatically so as to modify the light originating from the colored light source F into a uniform pattern in the irradiation window, or on the region 3a of the object 3 to be irradiated. The CCD camera then records the projected pattern, which is generally distorted due to the curvature of the surface 3a, and this can be stored as a basis for the subsequent spatial imaging on the screen D.

FIG. 5a shows a variant of the invention according to FIG. 5, wherein a DMD unit D is used instead of the LCOS unit D of FIG. 4a.

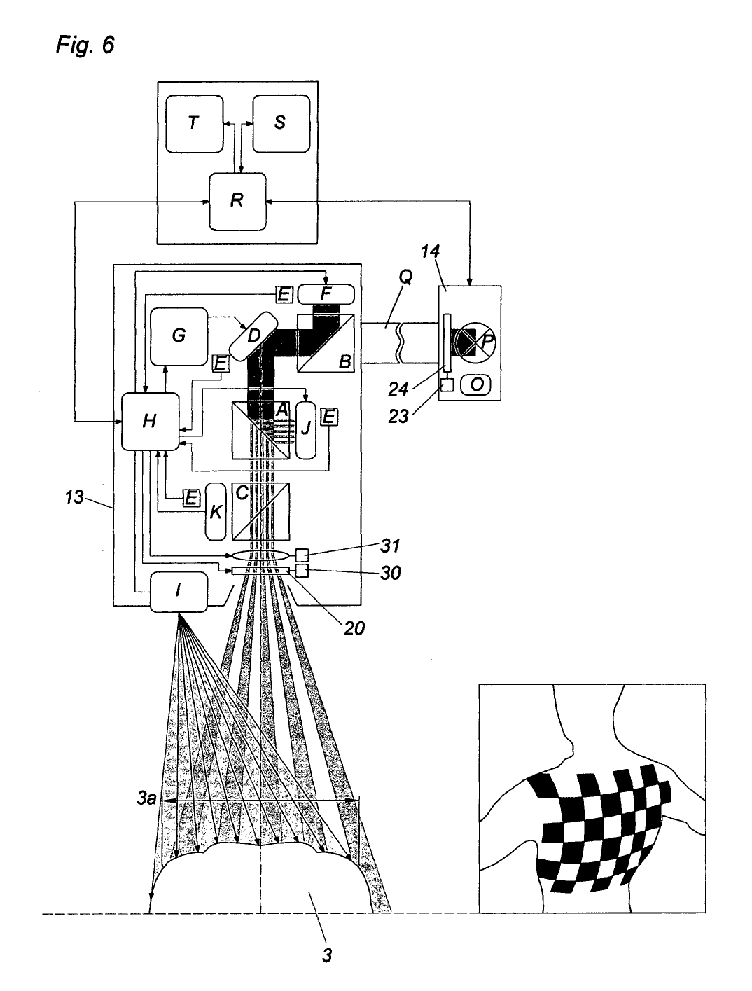

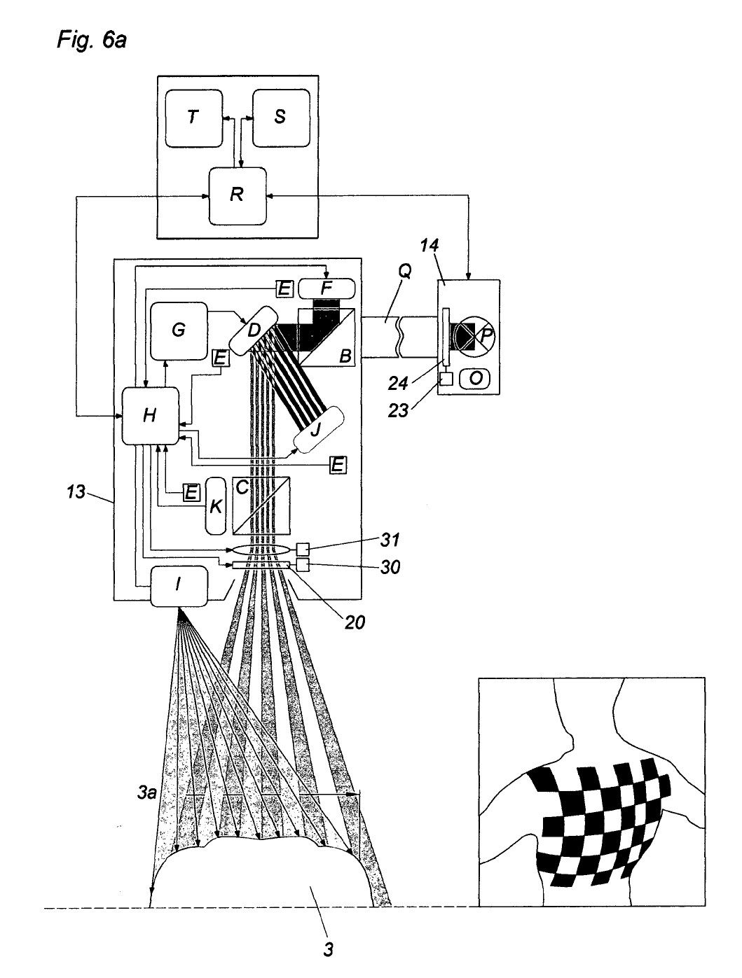

In the procedural step shown in FIGS. 6 and 7, in essence the sizes and positions of the individual partial regions A1 to A7 being irradiated, which vary according to the spatial structure of the surface 3a, are taken into consideration (see FIG. 7), and are then compensated for by computer. If the energy is known which is emitted within the solid angle region a of a partial surface to be irradiated, then in order to know the medically relevant intensity (i.e. energy per surface and time) it is necessary to know the surfaces of the individual partial regions A1 to A7 which generally vary for each partial region, since they are at different distances away from, and also have different orientations or positions in relation to, the irradiation head.

In order to detect these individual partial surfaces A1 to A7, shown diagrammatically in FIG. 7, there is provided a position detection device I for contact-free detection of the spatial course of the region 3a of the surface of the object 3 to be irradiated. The position detection device 3 is preferably arranged in, or on, the irradiation head 13, and thence it measures the surface 3a. In a preferred embodiment, the position detection device has a 3D-laser scanner for detecting the surface geometry of the object. The position detection device 3 can, however, also contain a means for projecting predefined patterns onto the object which are then captured by a camera and electronically evaluated.

The position detection device I is activated by means of an electronic control device R which evaluates the measured signals and possibly stores them.

The 3D laser scanner I thus measures the surface region covered by the irradiation window, and it transmits its data to the control software in the control computer R via the control unit H. A spatial model of facets of the surface region 3a, as covered by the imaging optics 20 of the irradiation head 13 and irradiation window, is calculated. Together with the distorted image detected by the CCD camera K according to FIG. 5, a 3D-correction matrix is set up by the control software, in which a partial region of the surface to be irradiated, or a corresponding solid angle region, corresponds to each field or element of the matrix. The values in the 3D correction matrix are correlated to the position or size of the surfaces A1 to A7 (see FIG. 7).

FIG. 6a, in turn, shows the DMD alternative to the LCOS variant of FIG. 6.

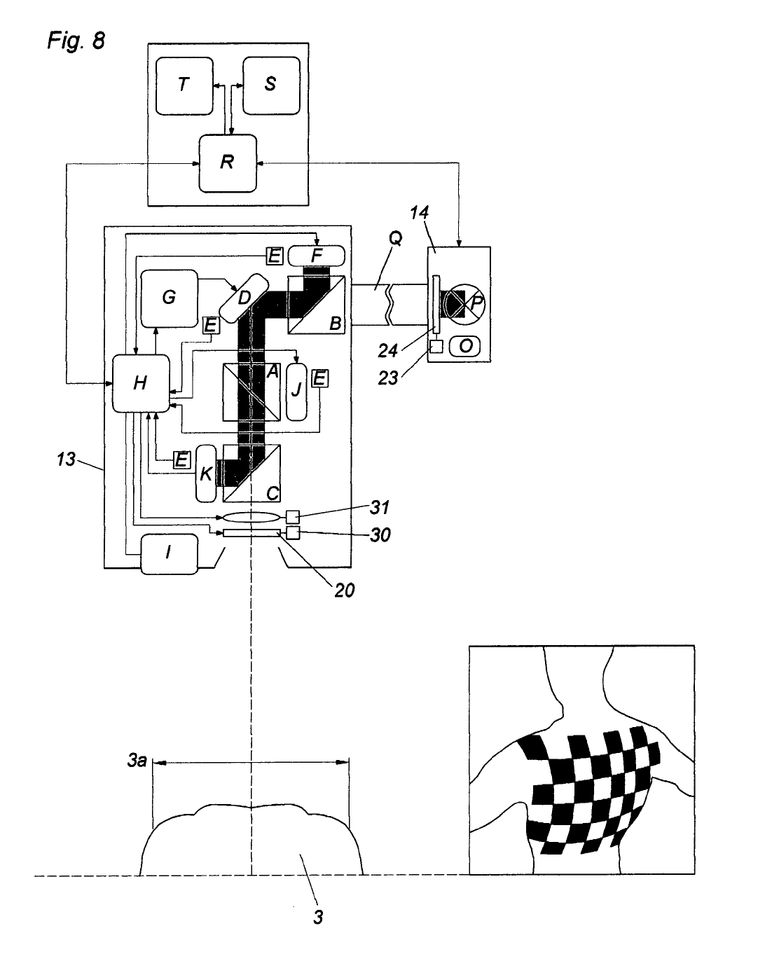

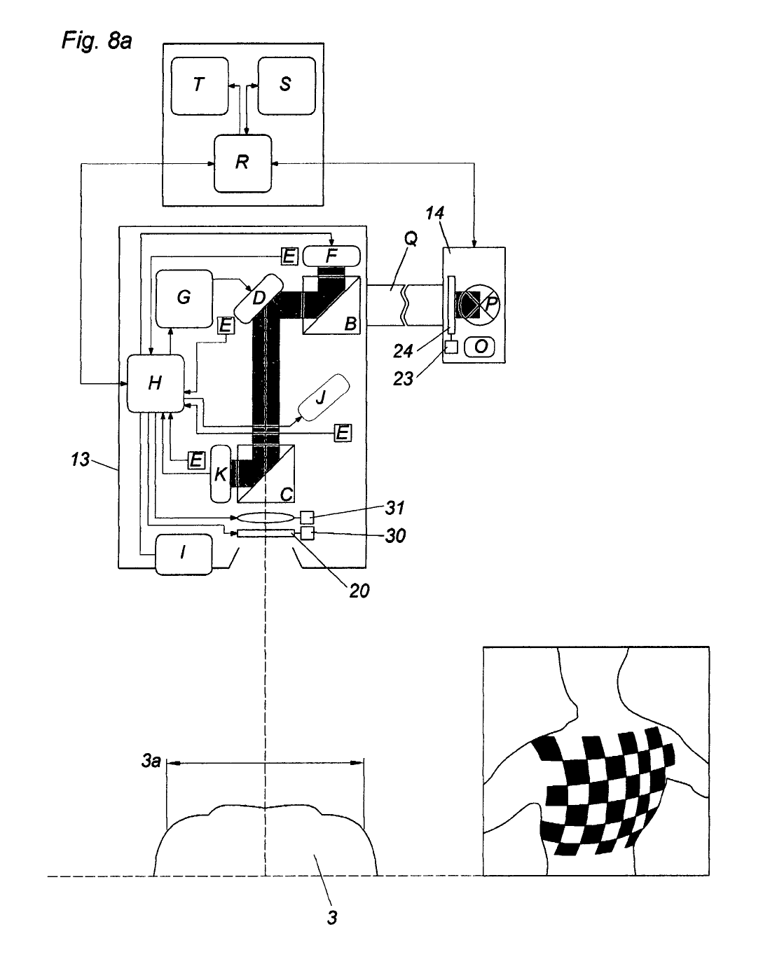

According to the mode in FIG. 8, calibration of the RGB colored light source F and of the CCD camera K can then take place as the next step.

To that end, the shutter 30 of the irradiation head 13 is closed by means of the motor 31, in order to be able to calibrate the CCD camera K. The camera A transmits a dark image to the control unit H. The RGB unit F is then programmed to emit white light. In this calibration stage, the prism C is pivoted through 90° (as shown in FIG. 8), so that the light reaches the camera K from the light source F directly via the modulator (i.e. it is not reflected by the object 3). The camera K then emits an image to the control unit H which then calculates a correction matrix which is temporarily valid for the duration of the treatment session, for any image distortion due to dust or scratches. At the same time, the control unit H calculates a correction matrix which optimizes a non-uniform illumination by the light source F by way of appropriate correction modulation of the modulator D into uniform light distribution through the projection window. In this step, any lack of uniformity of the light source F, or other optical components, can thus be compensated for, stored and then corrected.

FIG. 8a shows the DMD alternative to the LCOS variant of FIG. 8.

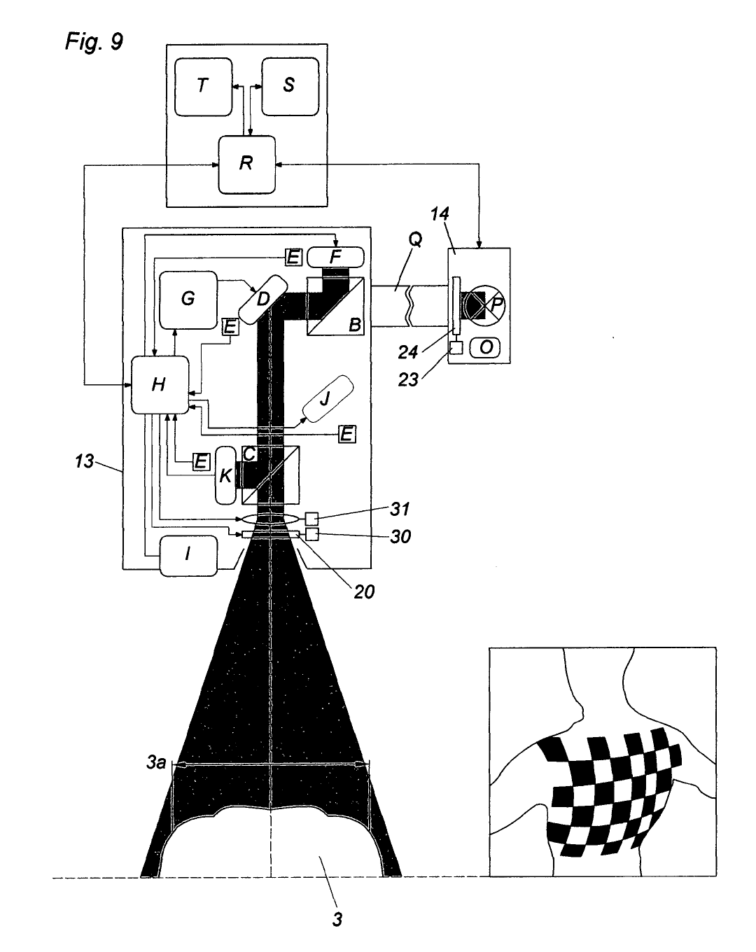

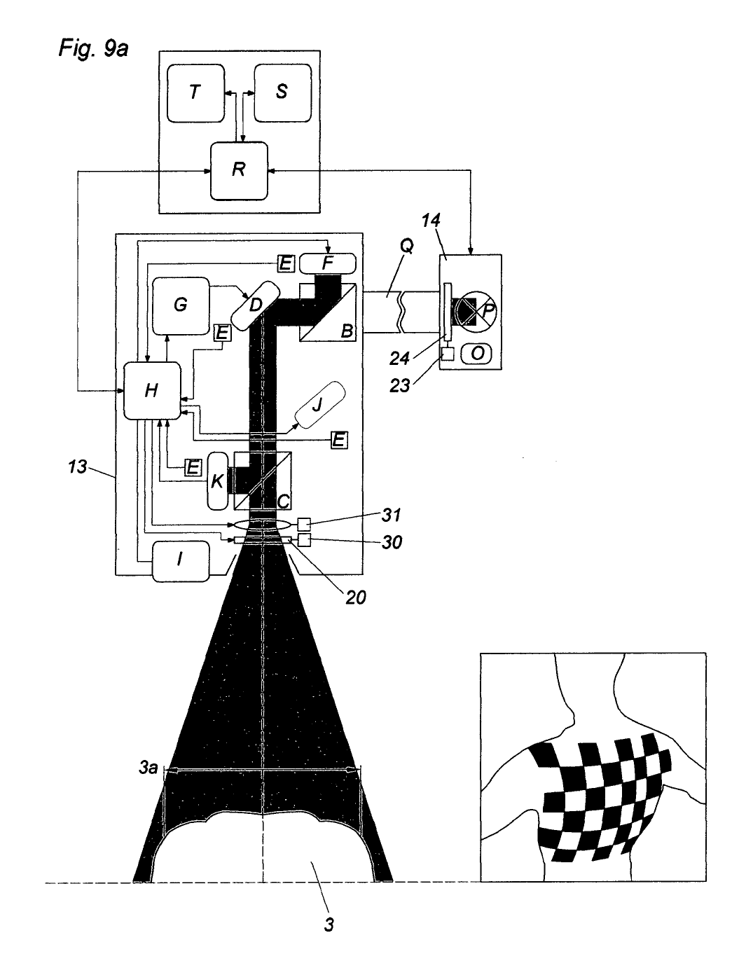

In the mode shown in FIG. 9, visible image acquisition takes place for the operator, e.g. for the doctor. Via the RGB light source F and the modulator D, the irradiation head 13 emits white light (calibrated in accordance with the earlier step) onto the entire irradiation surface 3a. Using that illumination, the camera K takes, for example, a number of color images per second of the surface to be irradiated, and transmits this series of images by means of the control unit H to the control software in the control computer R. By means of the software, the light intensity of the colored light source F can be set so that an optimally illuminated and thus assessable image of the skin surface is available within the control system for further processing.

FIG. 9a shows the DMD alternative to the LCOS variant of FIG. 9.

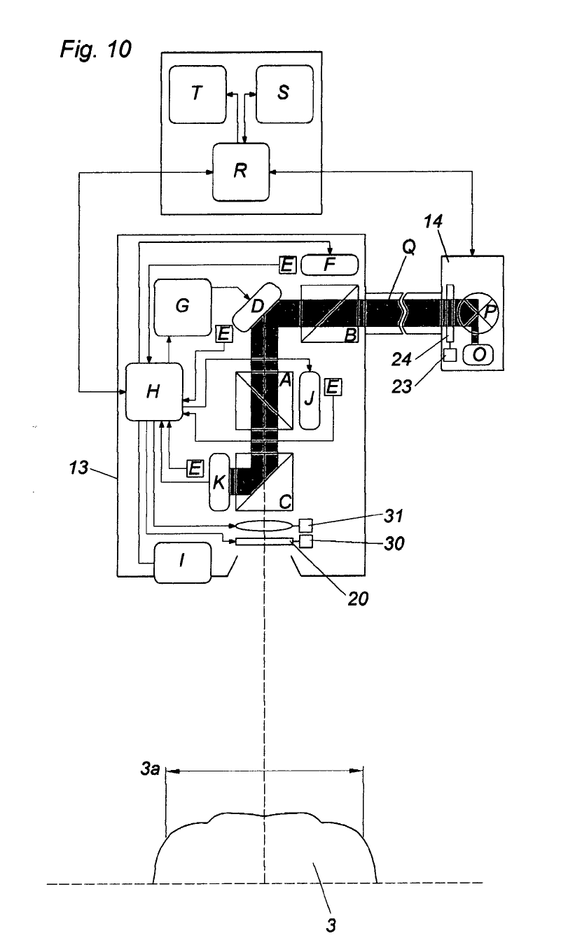

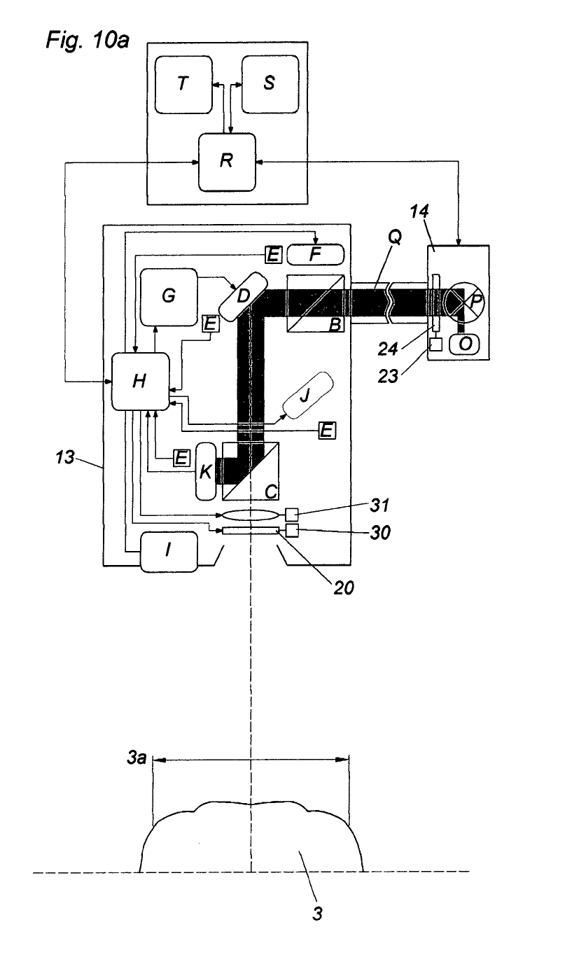

According to FIG. 10, before the start of the treatment, all parameters are once again scanned by the control software in the control computer so as to be passed by the operator. The RGB light source F is then deactivated, and by virtue of the control unit H the CCD camera K then takes on the task of adapting the radiation intensity to the individual image pixels (physical image elements of the modulator D). To that end, the shutter 30 of the irradiation device is closed by means of the motor 31, and the exit shutter 24 of the external UV light source P is opened by means of the motor 23.

The control software in the control computer R then alters the radiation intensity from 0% to 100% of the maximum radiation intensity calculated, and the CCD camera sends those images to the control unit H. This latter then forms from all of the image information collected and stored a two-dimensional correction mask (linearization) in the form of a grayscale picture which is offset with the previously defined medical irradiation mask (nominal values of intensity for the individual partial regions of the object) in such a way that the correct modulation images in the exact physical resolution of the modulator D across the modulation function (time/intensity) correspond integrated over each image point to the predetermined dose of radiation.

Before the start of the actual treatment, the photospectrometer O checks whether the defined wavelength-bandwidth exists.

FIG. 10a shows the DMD alternative to the LCOS variant of FIG. 10.

Before the actual treatment—i.e. before the irradiation with UV light begins—the doctor, or operator, in general terms, establishes the desired nominal values of intensity for the individual partial regions of the object. This can be done, for example, on the basis of patient data files which have been stored beforehand. However, it can also be done directly on the screen, e.g. by marking with a pen. On the screen, the doctor has a visible image of the patient's skin, and he can easily identify the regions to be treated. By way of the RGB light source, in parallel to this, the region which is identified on the screen and which is to be irradiated can be projected onto the skin, and thus monitored at the same time.

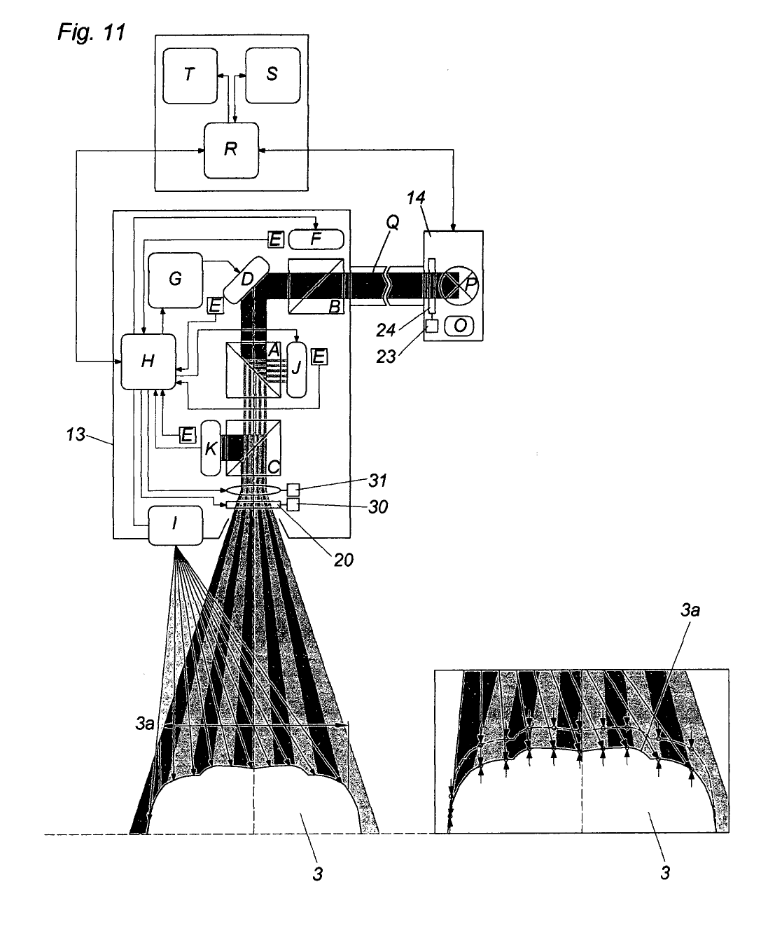

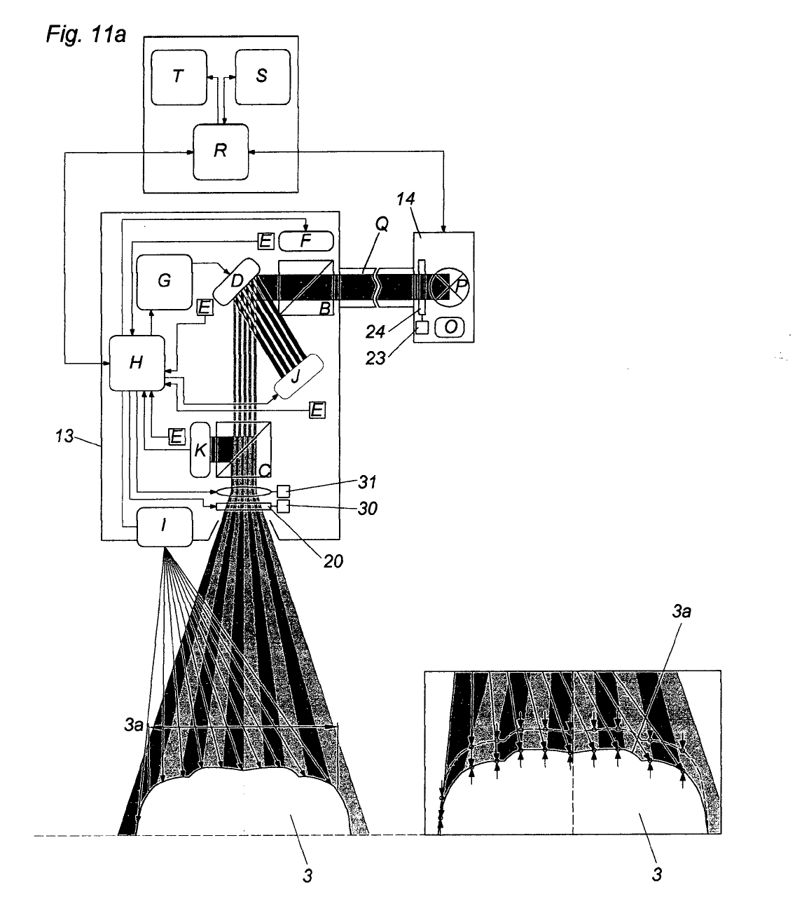

Since the irradiation device shown always knows the position of the individual partial regions by virtue of the position detection device, it is thus possible to use the control computer R, or control unit H, for the purpose of controlling the modulator D in such a way that the radiation output of UV light emitted from the irradiation head into the region of the solid angle corresponding to the partial region in question on the surface of the partial region of the object essentially gives the respective nominal value of intensity desired. In other words, the doctor, or operator, does not need to worry about the position, or distance away, of the object, not even if it sometimes changes due to breathing, as shown diagrammatically in FIG. 11, bottom right. If, for example, a partial region of the surface assigned to a specific solid angle region moves away from the irradiation head, and thus increases in size in terms of its surface area, the modulator compensates for this by supplying an appropriately greater amount of energy into that solid angle region, so that the desired nominal value of intensity is obtained on the surface of the skin.

FIG. 11a shows the DMD alternative to the LCOS variant of FIG. 11. The active irradiation procedure is shown in greater detail in FIG. 11, where it can be seen that parallel to the UV light the 3D laser scanner constantly monitors the position of the object.

Obviously, the invention is not restricted to the embodiments which have been shown. Numerous modifications are conceivable and possible within the scope of the claims.

1. A device for irradiating an object, in particular human skin, with UV light, having a UV light source and an irradiation head which contains imaging optics from which UV light is projected onto the object, wherein in addition to the UV light source a light source which emits visible light is provided, the light of which can be projected onto the object via the imaging optics of the irradiation head, wherein arranged in the irradiation head is a means which is preferably electronically controlled for adjustably setting the light distribution on the object, and that means can optionally or simultaneously be subjected to UV light from the UV light source and/or to visible light from the light source which emits visible light.

2. A device according to claim 1, wherein colored light and/or white light can be emitted by means of the light source which emits visible light.

3. A device according to claim 2, wherein the light source which emits visible light comprises an RGB-unit which preferably contains light diodes.

4. A device according to claim 1, wherein the light source which emits visible light is controllable by an electronic control unit, wherein the light intensity and/or the light color is adjustable.

5. A device according to claim 1, wherein the means for adjustably setting the light distribution on the object comprises an electronically controllable modulator for spatial light (EASLM).

6. A device according to claim 5, wherein the electronically controllable modulator for spatial light (EASLM) has a Digital Micromirror Device (DMD) or a Liquid Crystal on Silicon Unit (LCOS).

7. A device according to claim 1, wherein a visible color pattern—preferably in the form of a regular grid—can be projected onto the object (FIG. 4) by the means for adjustably setting the light distribution on the object.

8. A device according to claim 1, wherein an electronic control device is provided which comprises a screen, and that a visible image which is correlated with the current display on the screen can be projected onto the object by the means for adjustably setting the light distribution on the object, appropriately controlled by the electronic control unit.

9. A device according to claim 8, wherein the screen and the region of the object to be irradiated are offset alongside each other or one behind the other so that they can both be viewed from the same position of observation.

10. A device according to claim 1, wherein arranged in the irradiation head there is a camera, preferably a CCD camera.

11. A device according to claim 10, wherein the camera is configured in such a way that it converts into corresponding electrical image signals UV light emitted by the UV light source and/or visible light emitted by the visible light emitting light source.

12. A device according to claim 11, wherein the camera detects light which comes directly from the light source (FIGS. 8, 10) and/or light which is reflected by the object (FIGS. 5, 11).

13. A device according to claim 12, wherein an irradiation head, a switch-over device, preferably a rotatable beam-splitter, is provided, by means of which light which comes directly from the light source or light which is reflected by the object, can be deflected onto the camera.

14. A device according to claim 1, wherein arranged in or on the irradiation head is a means for detecting the distance away and/or spatial course of the surface of the object.

15. A device according to claim 1, wherein the UV light source is situated in a separate light source housing outside the irradiation head, and located between the light source housing and the irradiation head is at least one flexible fiber optic cable, via which UV light can be fed from the UV light source to the irradiation head.

16. A device according to claim 15, wherein the flexible fiber optic cable contains at least one quartz glass fiber.

17. A device according to claim 15, wherein the flexible fiber optic cable is enclosed in a light-proof manner.

18. A device according to claim 15, wherein the flexible fiber optic cable is connected by means of a releasable connection to the UV light source housing and/or the irradiation head.

19. A device according to claim 1, wherein provided in the light source housing, at the output side, and in the irradiation head, at the input side, are respective collimating lenses for respectively inputting and outputting UV light into and out of the fiber optic cable.

20. A device according to claim 1, wherein the irradiation head is adjustably mounted on a carrier device.

21. A device according to claim 20, wherein the irradiation head is displaceably and/or rotatably mounted.

22. A device according to claim 21, wherein the irradiation head is adjustable by means of a motor.

23. A device according to claim 1, wherein the object is adjustably mounted, preferably rotatably mounted about a vertical axis, on an object carrier device.

24. Use of a device according to claim 1 for irradiating human skin.
